(19) United States
(12) Patent Application Publication
(10) Pub. No.: US 2006/0046281 A1
(43) Pub. Date: Mar. 2, 2006

(54) Non-destructive method and test kit for the detection of infestation of live trees by invasive wood boring insect species

(76) Inventors: Georgette Smith, Fredericton (CA); Ken Harrison, Fredericton (CA)

Correspondence Address: NATIONAL RESEARCH COUNCIL OF CANADA;1500 MONTREAL ROAD, BLDG M-58, ROOM EG12, OTTAWA, ONTARIO, K1A 0R6, CA

(21) Appl. No.: 11/213,710

(22) Filed: Aug. 30, 2005

Related U.S. Application Data
(60) Provisional application No. 60/605,522, filed on Aug. 31, 2004.

(51) Int. Cl. C12Q 1/04

(52) U.S. Cl. 435/34

(57) ABSTRACT

The invention disclosed is a method and test kit for the non-destructive detection of infestation of live conifer trees by invasive wood boring insects having a specific established fungal associate. The method involves identifying an infested tree, providing a probe for collecting viable fungal spore samples and matching the spore sample with a known insect.

This application claims priority on U.S. Provisional Application 60/605,522 which was filed Aug. 31, 2004

### BACKGROUND OF THE INVENTION

This invention relates to a method and test kit for the non-destructive detection of infestation of live conifer trees by invasive wood boring insect species.

The early detection and accurate delineation of a quarantine invasive species is critical to Regulatory Programs. The earliest detection of an invasive species is by confirmation of its presence in or on the living host tree. This is particularly challenging at locations where the infestation is at low, endemic levels.

Current (conventional) methods may result in false negatives and may require destructive sampling by the removal of bark or cutting the tree to procure a wood sample(s).

Currently, detection of the invasive species on the tree relies entirely on hand picking suspects that are visible on the trunk of the tree. The detection of the invasive species on the host tree by hand picking of the pest is seasonal and restricted to a very narrow time frame of a few weeks. It is time and labour intensive and limited to suspects within the field of vision, so if a suspect is not in the field of vision of the inspector, a false negative may result. If the tree is infested and not cut, it becomes the source of a subsequent infestation, and this does not meet the objective of the Regulatory Program.

The detection of a life stage of the invasive species in the tree is less seasonally dependent, but it is time and labour intensive and destructive to the suspect tree. The tree may subsequently prove to have been free of the pest (false positive). Although the objectives of the Regulatory Program may have been met, the unnecessary cutting of healthy, uninfested trees is generally unacceptable, as it can be the source of strong public objection or result in legal issues.

Pivotal to the success of the present invention is an understanding of both the morphology of the exit hole in the tree from which the adult insect emerges and the intricacies of the design of the connected exit tunnel and pupal cell in the wood. This construction is excavated by the prepupal larva, which has ceased feeding and producing frass. The result is a clean, moist, wood environment highly suitable to the proliferation of fungal fruiting bodies bearing spores during larval, pupal, and adult insect occupation. Fungal spores are borne on the dense hairs on the body of the emerging adult insect, which becomes a vector for the fungus infection of further trees.

The invasive quarantine species Tetropium fuscum (Fabr.), the brown spruce longhorn beetle (BSLB), is an example of such invasive wood boring insect species, and follows this scenario, and live spores of the associated blue-stain fungus, Ophiostoma tetropii, have been isolated consistently on selective media from T. fuscum pupal cells and from the living insect (Smith and Harrison, unpublished data) and the presence of this fungus is therefore considered a positive indicator of the presence of its insect vector (Harrison et al, 2004¹).

The brown spruce longhorn beetle, Tetropium fuscum (Fabricius) (Coleoptera: Cerambycidae), is established in and around Point Pleasant Park, a 75-ha heritage park in the Halifax Regional Municipality Nova Scotia. It has also been intercepted in solid wood packaging at the Canadian ports of Montreal and Vancouver. This woodborer is native to northern and central Europe and western Siberia where it typically attacks dead and dying trees. In Halifax, T. fuscum is primarily attacking living, apparently healthy red spruce (Picea rubens Sarg.), the predominant spruce of the Park. Other trees attacked include black spruce (P. mariana (Mill.)BSP), white spruce (P. glauca (Moench) Voss) and Norway spruce (P. abies (L.) Karst).

This is the first established population of T. fuscum discovered in North America. The geographic range of the native spruce (Picea) species attacked in Halifax indicates a potential threat to the predominant tree species of northern coniferous forests. Red spruce is restricted to northeastern North America; however, black and white spruce range across Canada in the boreal forest.

Three methods are currently used for fungal spore collection. They all require destructive sampling of a living, standing tree assessed as healthy and they produce different rates of success.
1) All BSLB pupal cells examined since 2000 have contained O. tetropii fruiting bodies, yielding a 100% success rate.
2) Culturing from the live insect vector produces a success rate of ˜90%.
3) The traditional technique of culturing directly from the sapwood of the suspect tree can produce false negative results.

### SUMMARY OF THE INVENTION

In solving the problems posed by the three above mentioned conventional methodologies, we produced and began lab testing of a novel, non-destructive method and test kit for the early detection of infestation of live conifer trees of an invasive quarantine insect species by exploiting the physical properties of its excavations in the host tree, specifically the morphological features of the insect pupal cell and associated structures (exit tunnel and exit hole). The procedure is non-destructive and results should be comparable to collecting spores directly from fruiting bodies in the pupal cell.

Thus, the methodology and test kit according to the invention can be utilized for both the early detection of invasive wood boring insects having specific established fungal associates, and the early detection and identification of fungal species pathogenic to live conifer trees. Definitive and rapid diagnosis is critical to the early detection and identification of invasive species, some of which are of quarantine significance.

According to one aspect of the present invention, a non-destructive method is provided for the detection of infestation of a living conifer tree by a wood boring insect having a specific established fungal associate, comprising
(a) identifying an infested tree by the visual presence of insect exit holes of a characteristic shape, size and orientation in the bark and by a characteristic pattern of visible resin flow not originating from the insect exit holes, or from visible wounds e.g. from frost cracks or mechanical injury.
(b) providing an elongated sterile probe having a diameter and length to accommodate the range of diameters of insect exit holes and length of connected insect exit tunnels i.e., a diameter less than that of the exit holes, and having a fungal spore collecting tip,
(c) inserting the probe into an exit hole to collect viable fungal spores or mycelium,
(d) withdrawing the probe from the exit hole, and securing it in a receptacle containing a fungus friendly medium,
(e) matching the fungus with a wood boring insect species known to have a specific established fungal associate.

In one embodiment of this aspect of the invention, step (e) includes culturing viable fungal spores or mycelium that are present on the probe tip.

Since some fungi do not fruit readily or reliably in pure culture, including many “blue stain” fungi e.g., ophiostomatoid species, diagnosis is delayed or otherwise impaired. Accordingly, in another embodiment of this aspect of the invention, step (e) includes removing the viable fungal spores or mycelium from the probe, and extracting fungal DNA therefrom.

The extraction method, is a classical method, including depositing the spores or mycelium on a glass slide, crushing them between the glass slide and a cover glass, and conducting PCR from the resulting liquid. See Zambino, P. J. and Szabo, L. J.⁵, the disclosure of which is incorporated herein by reference.

According to another aspect of the invention, a test kit is provided for the detection of infestation of a living conifer tree by a wood boring insect having a specific established fungal associate, comprising an elongated sterile probe having a diameter and a length characteristic of the dimensions of the target insect exit holes and exit tunnels, and a fungal spore collecting tip, and a set of instructions for identifying an infested tree by the visual presence of insect exit holes of a characteristic shape, size, and orientation in the bark and by a characteristic pattern of visible resin flow not originating from the insect exit holes or from visible wounds (i.e., frost cracks or mechanical injury) and for using the probe to collect viable fungal spores or mycelium.

The trademark MorphoProbe™ is associated with the test kit.

### BRIEF DESCRIPTION OF THE DRAWING

FIG. 1 is a perspective view of one embodiment of a probe used in the invention.

FIG. 2 is a perspective view of another embodiment of a probe used in the invention.

### DETAILED DESCRIPTION OF THE INVENTION

Materials and Methods:

In one embodiment the test kit includes an elongated probe, provided in a pre-sterilized unit with a thin handle and a pre-moistened tip which is secured in a receptacle containing a fungus friendly medium to maintain viability of the fungus. This provides a reliable delivery system for the optimum collection of viable fungal spores while minimizing the risk of contamination, which is inherent to culturing in the field. The lightweight, readily hand portable device, once removed from its receptacle, can be gripped in one hand and readily inserted in the insect exit hole. Following the orientation of the exit tunnel in the wood, the probe will slide along until the tip makes contact directly with the pupal cell and/or its juncture with the exit tunnel. The probe, bearing viable fungal spores at its tip, is easily retracted, returned to its receptacle, and is ready for transport to the lab for culturing and growth on a selective, nutrient rich medium appropriate to the fungal species for maintaining viability.

To accommodate the range of diameters of exit holes and configuration of exit tunnels of T. fuscum (BSLB) and other insects having exit holes of the same or similar morphology, including longhorn beetles (Cerambycidae), bark and ambrosia beetles (Scolytidae), weevils (Curculionidae), and wood wasps (Siricidae), the insect groups containing the majority of quarantine (regulated) species, two variations of the probe have been developed.

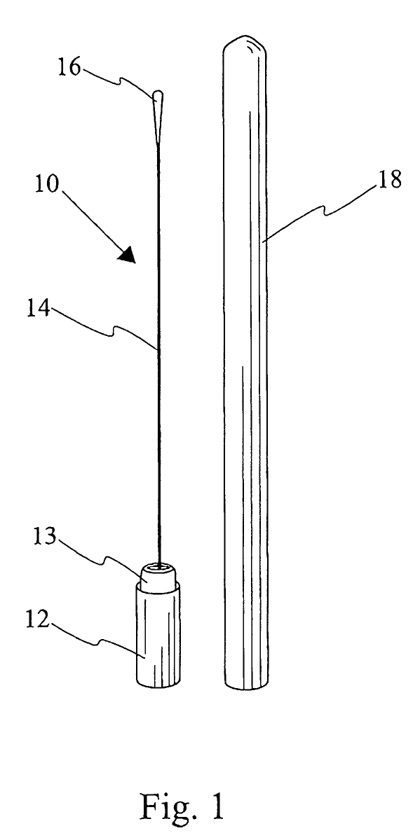

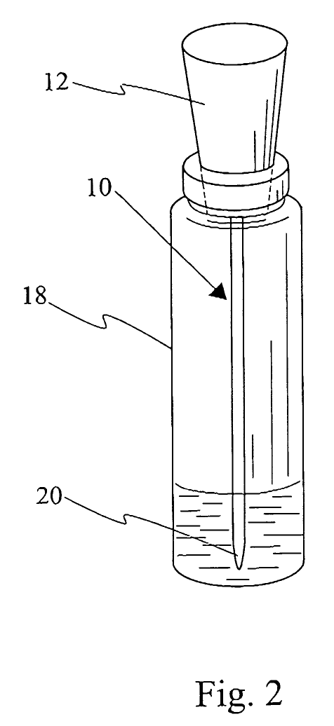

1). A unit (culturette), intended for human medical purposes is commercially available through Fisher Scientific, comprising a probe 10, having a handle 12 including a reduced diameter extension 13 for receiving a receptacle 18 and a long (6.5 mm) flexible wire shaft 14 having a small diameter (2.5 mm), moist, swab tip 16 (FIG. 1), can be readily employed in sampling spores via the exit holes of BSLB, other longhorn beetles, and large bark beetles, as it accommodates their range of exit hole diameters (3.0 mm or more) and depths of exit tunnels (0.4 cm to 4.0 cm). The receptacle 18 is provided to enclose the probe 10, and when seated in the handle in a telescoping manner, shields the probe from the atmosphere, and provides an enclosed sterile environment. The seating may be effected e.g by complementary screw threads on the outside of extension 13 and on the inside of receptacle 18. Alternatively, the extension and receptacle could be dimensioned for a force-fit.
2) A unit intended exclusively for sampling via insect exit holes has been constructed entirely from materials readily available in the Taxonomy lab at CFS-Atlantic, Fredericton, NB, Canada. It comprises a unit sterilized (autoclaved) in-house including a receptacle 18, in the form of a 4-dram patent lip glass vial (21 mm diameter x 70 mm length), a #0 neoprene stopper 12 for closing the receptacle and shielding the probe from the atmosphere, and a probe 10 in the form of a wooden, cylindrical toothpick (length 6.5 cm, diameter 0.1 cm), tapered at both ends, one end of which is inserted to a depth of ˜2.0 mm in the bottom of the stopper. When fitted in the glass vial enclosure, the free tip of the probe 20 is immersed in 5.0 ml of sterilized water in the bottom of the vial (FIG. 2). It will be appreciated that other receptacle enclosures could be used, such as a screw-threaded cap. The water is discarded during sampling, providing a moist chamber conducive to spore preservation during transport and storage, once the spore-bearing probe is retracted from the exit hole and replaced in the receptacle. This unit can be readily employed in sampling via exit holes of a wide range of dimensions.
   The test kit also includes a set of instructions for identifying an infested tree by the visual presence of exit holes of a characteristic size, shape, and orientation in the bark and by a characteristic pattern of visible resin flow not originating from the insect exit holes (Smith and Humble, 2000²) or from visible wounds (i.e., frost cracks or mechanical injury).

More specifically, trees infested with BSLB exhibit round to oval shaped exit holes averaging about 4 mm in diameter, and produce excessive amounts of resin flow streaming down the length of the trunk in a characteristic pattern. The exit holes connect by tunnels with a pupal chamber and an extensive network of feeding tunnels up to 6 mm across.

Results/Discussion:

Preliminary results of lab testing indicate a success rate of 98%-100% (ρ=100+) for both forms of the probe.

Preliminary field tests in June 2004, indicate that the long, thin flexible wire handle of the commercially available probe (Method 1) can be impractical and that the CFS-constructed MorphoProbe (Method 2), with its stiffer, non flexible and easily controlled construction is more feasible for field use.

Example on Spruces:

An invasive alien species of wood boring beetle, Ips typographus (L.), (Coleoptera:Scolytidae) “the European spruce bark beetle” is not yet known to be established in North America but is of the highest priority (A1) on the quarantine list for this continent. This insect is a two-pronged threat to North American spruce trees since in addition to the damage from insect infestation, it can introduce several species of blue-stain fungi (Ceratocystis polonica and several Leptographium spp.). These blue-stain fungi (especially C. polonica) cause a noticeable deep staining of wood products and its presence would be a direct threat to wood exports (non-tariff trade barrier). Ceratocystis polonica is pathogenic to trees and is known to be vectored by Ips typographus. See Kirisits, T.⁶ The spores of one or more of these fungal species could be collected with the Probe via the exit hole in the living spruce tree and thus provide early and non-destructive indication of the presence of the quarantine insect.

Example on Pines:

Leptographium wingfieldii is a fungal associate of several invasive alien scolytid species (Hylastes opacus, Hylurgops palliatus and Tomicus piniperda “the European pine shoot beetle” and it has recently been found associated with several species of our native bark beetles in Ontario and the northeastern United States. The presence of Leptographium wingfieldii would be a strong indication that one or more of the alien insect species have spread and become established in new areas. See Jacobs, K. and Wingfold, M. J.⁷

To date, field testing has provided a success rate for detection of 90-92%. See below.

This early detection tool (MorphoProbe) can be applied to a wide array of wood boring insects with known fungal affiliates, including the following taxonomic groups:
Cerambycidae (longhorn beetles)
Scolytidae (bark and ambrosia beetles)
Curculionidae (weevils)

The association between weevils and Leptographium procerum (the cause of white pine root decline) is documented in: Jacobs, K. and Wingfield, M. J.⁷.

Jacobs and Wingfield⁷ also make specific reference to Leptographium procerum being associated with the Pissodes spp. weevils (Coleoptera: Curculionidae), in particular Pissodes approximatus, P. nemorensis and P. pini and provide 6 references.

The association between Siricidae (wood wasps or horntails) and an Amylostereum spp. Fungus is documented in Viitasaari, M. and Heliovaara, K.⁸:

The blue stain and wood decay fungi associated with certain insect species are pathogenic to trees, e.g., Leptographium terebrantis affiliated with the indigenous North American species Dendroctonus valens (Coleoptera:Scolytidae) (Wingfield³, 1986; Owen⁴ et al, 1987).

This problem can be overcome by the use of fungal DNA for rapid and definitive diagnostics. Good quality fungal DNA can be extracted from viable spores and used for the differential diagnosis of the target fungal species and non-target fungi, with an average turn-around of results in 2 to 3 days. See Zambino, P. J. and L. J. Szabo⁵.

MorphoProbe could be submitted to a molecular diagnostics lab and fungal DNA could be extracted from viable spores or mycelium it has collected. This option has a two-fold advantage: 1) it provides rapid results for target fungal species that will not produce the definitive (fruiting) stage in pure culture and 2) it can dramatically reduce the time and labour for culturing target fungal species, e.g., Ophiostoma tetropii associated with Tetropium fuscum, the Brown Spruce Longhorn Beetle that do fruit readily in culture. Thus, the culturing step which adds an average of 3 weeks to the process, is avoided.

### REFERENCES

1. Harrison, K. J., Smith, G. A., Hurley, J. E. and MacKay, A. W. 2004. Ophiostoma tetropii as a detection tool for the brown spruce longhorn beetle, Tetropium fuscum (Fabr.), in Halifax, Nova Scotia. Canadian Plant Disease Survey, Volume 84:125-126.
2. Smith, G. A. and Humble, L. M. 2000. The Brown Spruce Longhorn Beetle. Exotic Forest Pest Advisory #5, Natural Resources Canada, Canadian Forest Service.
3. Owen, D. R., Lindahl, K. Q. Jr, Wood, D. L. & Parmeter, J. R. Jr. 1987. Pathogenicity of fungi isolated from Dendroctonus valens, D. brevicomis, and D. ponderosae to ponderosa pine seedlings. Phytopathology 77:631-636.
4. Wingfield, M. J. 1986. Pathogenicity of Leptographium procerum and L. terebrantis on Pinus strobus seedlings and established trees. European Journal of Forest Pathology 16:299-308.
5. Zambino, P. J. and L. J. Szabo. 1993. Phylogenetic relationships of selected cereal and grass rusts based on rDNA sequence analysis. Mycologia 85 (3):401-414.
6. Kirisits, T. 2004. Fungal associates of European bark beetles with special emphasis on the ophiostomatoid fungi (Chapter 10) in F. Lieutier et al. (eds.) Bark and Wood Boring Insects in Living Trees in Europe, A Synthesis, pp. 181-235. Kluwer Academic Publishers 2004. See Table 2, p. 207-209. Twenty-four references cited for Ceratocystis polonica alone on p. 207.
7. Jacobs, K. and Wingfield, M. J. 2001. Leptographium Species: Tree Pathogens, Insect Associates, and Agents of Blue-Stain. APS Press, St. Paul, Minn. p. 24. (cites 6 earlier papers)
8. Viitasaari, M. and Heliovaara, K. 2004. “8. Siricidae (Horntails)” Section 8 of Chapter 22 in F. Lieutier et al. (eds.) Bark and Wood Boring Insects in Living Trees in Europe, A Synthesis, pp. 501-538. Kluwer Academic Publishers 2004. pp. 530-532.

1. A non-destructive method for the detection of infestation of a living conifer tree by a wood boring insect having a specific established fungal associate, comprising
(a) identifying an infested tree by the visual presence of insect exit holes of a characteristic shape, size and orientation in the bark and by a characteristic pattern of visible resin flow not originating from the insect exit holes
(b) providing an elongated sterile probe having a diameter and length to accommodate the range of diameters of insect exit holes and length of connected insect exit tunnels, and having a fungal spore collecting tip,
(c) inserting the probe into an exit hole to collect viable fungal spores or mycelium,
(d) withdrawing the probe from the exit hole, and
(e) matching the fungus with a wood boring insect species known to have a specific established fungal associate

2. A method according to claim 1, wherein step (e) includes culturing viable fungal spores or mycelium that are present on the probe tip.

3. A method according to claim 2, wherein the wood boring insects are of a taxonomic group selected from the group consisting of Cerambycidae, Scolytidae, Curulionidae and Siricidae.

4. A method according to claim 3, wherein the wood boring insects are of a taxonomic group selected from the group consisting of Cerambycidae and Scolytidae.

5. A method according to claim 2, wherein the insect/fungus pairing is Tetropium fuscum and Ophiostoma tetropii.

6. A method according to claim 1, wherein step (e) includes removing the viable fungal spores or mycelium from the probe, and extracting fungal DNA therefrom.

7. A method according to claim 6, wherein the wood boring insects are of a taxonomic group selected from the group consisting of Cerambycidae, Scolytidae, Curculionidae and Siricidae.

8. A method according to claim 6, wherein the wood boring insects are of a taxonomic group selected from the group consisting of Cerambycidae and Scolytidae.

9. A method according to claim 6, wherein the insect/fungus pairing is Tetropium fuscum and Ophiostoma tetropii.

10. A test kit for the detection of infestation of a living conifer tree by a wood boring insect having a specific established fungal associate, comprising an elongated sterile probe of diameter and length to accommodate the range of diameters of insect exit holes and length of insect exit tunnels and a fungal spore collecting tip, and a set of instructions for identifying an infested tree by the visual presence of insect exit holes of a characteristic shape, size, and orientation in the bark and by a characteristic pattern of visible resin flow not originating from the insect exit holes and for using the probe to collect fungal spores or mycelium.

11. A test kit according to claim 10, further comprising a sterile tubular receptacle for containing the probe, having an open end closed to the atmosphere by a stopper.

12. A test kit according to claim 11, wherein the receptacle contains a medium for maintaining viability of the collected spores.

13. A test kit according to claim 12, wherein the medium is selected from the group consisting of sterilized water and a nutrient rich medium.
